(19) United States
(12) Patent Application Publication
(10) Pub. No.: US 2020/0199521 A1
(43) Pub. Date: Jun. 25, 2020

(54) ACOUSTIC ENERGY BASED CELL LYSIS AND NUCLEIC ACID FRAGMENTATION

(71) Applicant: Covaris, Inc., Woburn, MA (US)

(72) Inventors: James A. LAUGHARN, JR., Boston, MA (US); Hans-Ulrich Thomann, Stow, MA (US)

(73) Assignee: Covaris, Inc., Woburn, MA (US)

(21) Appl. No.: 16/713,145

(22) Filed: Dec. 13, 2019

Related U.S. Application Data
(60) Provisional application No. 62/781,717, filed on Dec. 19, 2018.

(51) Int. Cl. C12N 1/06; C12N 15/10; C12M 1/00

(52) U.S. Cl. CPC C12N 1/06; C12M 47/06; C12N 15/1003

(57) ABSTRACT

A method for lysing cells and shearing genomic DNA to reduce viscosity of the cell lysate. Cells may be lysed to release cell lysate, and the cell lysate may be treated with focused acoustic energy to shear genomic DNA so that the genomic DNA is sheared to DNA fragments having a fragment size no larger than 50% of the starting base pair size of the genomic DNA. Lysing and DNA shearing may be done by acoustic energy and in a single vessel, allowing for automated handling of cell lysate.

### RELATED APPLICATION

This Application claims priority under 35 U.S.C. § 119(e) to U.S. Provisional Application Ser. No. 62/781,717, filed Dec. 19, 2018, which is herein incorporated by reference in its entirety.

### BACKGROUND

### 1. Field of the Invention

Systems and methods for lysing cells and fragmenting nucleic acid, e.g., in a single vessel, are generally disclosed.

### 2. Related Art

Various techniques are known for lysing cells, e.g., to release genomic and other material from a cell, including chemical and mechanical processes that chemically and/or mechanically degrade or otherwise damage outer cell and compartmental cell membranes to release cellular contents (known as cell lysate). Also, various techniques are known for shearing genomic material, such as genomic DNA, once the genomic material has been separated or otherwise recovered from other components of the cell lysate.

### SUMMARY

Aspects of the invention provide a method and apparatus for lysing cells to release genomic material and shearing/fragmenting the high molecular weight (HMW) genomic material, such as DNA, to relatively smaller sizes, e.g., 50% or less of a starting base pair size, without separating the genomic material from the cell lysate. Simultaneously, the HMW material is disentangled/dissociated from other biomolecules such as RNA and proteins, so that the viscosity of the lysate is reduced, making these components available for downstream processing such as binding to matrices, etc. The genomic material may be suitably sheared for effective use in downstream analysis techniques, such as sequencing analysis, qPCR, etc. As a result, in some embodiments, cells may be lysed and released genomic material may be sheared and dissociated from other cell lysate material in a single vessel such that recoverable genomic material may be produced without removal of the genomic material or other cell lysate components from the vessel. Not only does the shearing of DNA make recovery of the DNA possible or improve yield of recovered DNA, the resulting reduction in viscosity of the cell lysate can enable the recovery of other biomolecules from the cell lysate, such as proteins. That is, cell lysate is highly viscous at least in part because of genomic DNA. The high viscosity can make pipetting of the cell lysate difficult or impossible. Focused acoustic energy treatment can reduce the viscosity of the cell lysate, at least in part by shearing the genomic DNA, which can allow pipetting of cell lysate for recovery of biomolecules from the cell lysate.

Moreover, the viscosity reduction caused by focused acoustic energy treatment of cell lysate can allow for automation of cell lysing and genomic DNA and/or other biomolecule recovery. For example, in a mild conventional lysing process (e.g., chemical or mechanical) that may be appropriate for certain classes of biomolecules (e.g., complex carbohydrates), the mild process may result in a viscous lysate to which focused acoustic energy could be applied to reduce viscosity. Specifically, a 96 well plate of cellular or tissue samples may undergo a physical-chemical freeze/thaw to crack the cell walls as a “coarse” lysis, and could be followed by use of focused acoustic energy as a “fine” lysis/viscosity reduction process. Highly viscous cell lysate is difficult to pipette by hand, and so is not amenable to automated pipetting operations because robotic or other automated pipetting operations can often fail to remove material from a vessel containing cell lysate. Thus, automated processing without a significant workflow hindrance such as centrifugation or vacuum passage of the sample through a column to reduce viscosity is not attempted because removal of cell lysate material cannot be done in a repeatable and consistent way. Additionally, passage through a column typically results in a significant sample loss and dilution. In other words, a simple one-vessel process is highly preferable. Embodiments of the invention allow for whole cells to be provided in a vessel, cells lysed to release cellular contents in the vessel, and shearing of genomic DNA in the cell lysate to reduce the viscosity of the cell lysate in such a way that automated pipetting of at least portions of the cell lysate from the vessel can be performed in a repeatable and consistent fashion so that the processing can be automated. To date it has simply not been possible to provide whole cells in a vessel, lyse the cells in the vessel, and pipette cell lysate from the vessel using automated processing in a reliable way. Embodiments of the invention allow for the possibility of automation which was before not possible. Also, the recovered biomolecules from a sample may be of a high enough yield and quality so as to be immediately suitable for high-throughput analysis (e.g., via NGS, PCR, digital droplet PCT, etc.). This is in contrast with conventional extraction methods that do not employ focused acoustics in the manner described herein. Such conventional extraction methods have been observed to result in the recovery of biomolecules having a comparatively lower yield (e.g., recovery by mass) and lower quality (e.g., a lower percentage of DNA which is PCR amplifiable), which is unsuitable for certain types of high-throughput analysis, such as NGS, accurate and repeatable nanoliter droplet size distribution and analysis.

Other advantages and novel features of the present disclosure will become apparent from the following detailed description of various non-limiting embodiments when considered in conjunction with the accompanying figures and claims. For example, while the extraction of certain types of biomolecules is discussed herein, a variety of different compounds may be recovered from a blood sample, including metabolites and/or other compounds included in blood.

### BRIEF DESCRIPTION OF THE DRAWINGS

Aspects of the present disclosure are described with reference to the following drawings in which numerals reference like elements, and wherein:

FIG. 1 illustrates DNA yields in a comparative example in which whole blood cells are treated with focused acoustic energy or vortexing to lyse blood cells;

FIG. 2 illustrates DNA fragment sizes for the comparative examples of FIG. 1;

FIG. 3 shows the DNA yield for experiments in which whole blood was treated with varying levels of focused acoustic energy in comparison with a sample treated by repeated pipetting;

FIG. 4 shows electropherograms of DNA fragments from three samples treated with focused acoustic energy from FIG. 3;

FIG. 5 shows a correlation of Ct values and DNA input for an experiment involving lysis of yeast cells using different total treatment times with focused acoustic energy;

FIG. 6 illustrates the total energy used to achieve particular DNA fragment modes when treating isolated DNA and whole cells with focused acoustic energy;

FIG. 7 shows DNA and RNA yields when employing a commercial RNA purification kit both with and without the use of focused acoustic energy to lyse and homogenize cells; and

FIG. 8 shows a schematic block diagram of an acoustic treatment system that incorporates one or more aspects of the present disclosure;

### DETAILED DESCRIPTION

Aspects of the present disclosure are not limited in application to the details of construction and the arrangement of components set forth in the following description or illustrated in the drawings. Other embodiments may be employed and aspects of the present disclosure may be practiced or be carried out in various ways. Also, the phraseology and terminology used herein is for the purpose of description and should not be regarded as limiting.

Lysis of cells is a pre-requisite of biomolecule extraction. During cell lysis, the cell walls and membranes are broken and the contents of the cytoplasm leak into the lysing matrix. Lysis can be done by purely mechanical means such as microgrinding (bead beating), via a Douncer, or by chemical means (osmotic pressure, detergent-based buffers and or organic solvents such as phenol). Often, cell lysis is done by a combination of mechanical and chemical conditions to facilitate the extraction and stabilization of the biomolecules of interest. The combination of mechanical and chemical lysis is fundamentally different from purely chemical lysis methods, as the mechanical force assists in the homogenization of the lysate. Purely chemically obtained cell lysates are often very viscous, especially when a higher cell count is present in the sample. The viscosity is mostly due to high molecular weight (HMW) DNA, i.e., genomic DNA, that is present along with other cell lysate components such as cell debris, proteins, glycosides, and lipids.

Mechanical homogenization of cell lysates is commonly done via bead beating. Bead beating uses a grinding media in the form of small beads (zirconium oxide, steel, glass etc.) which are added to a sample containing whole cells and then shaken at a high velocity. This process creates friction and generates heat from bead collisions which can destabilize and/or degrade certain biomolecules. Moreover, the bead beating process is not automatable, e.g., because the resulting cell lysate material is too viscous to handle with automated pipetting equipment.

The inventors have found that use of focused acoustic energy to lyse cells to release cellular contents and reduce the viscosity of cell lysate provides superior results in terms of biomolecule recovery yields and quality, as well as provides an automatable process. Focused acoustic energy can lyse the cell wall and membranes of whole cells as well as homogenize cellular components, such as protein complexes, ribosomes, cell membrane structures, and, in case of eukaryotic cells, organelles, such as the nuclear compartment and chromatin. Proper homogenization is important for reproducible downstream processing, such as binding of analytes to purification matrices (silica, carboxylated silica, ion-exchange matrices, etc.) and reproducible transfer of cell lysate from the lysing vessel for direct measurements/assays such as qPCR, digital PCR, ELISA assays, immunoprecipitation etc. Lack of sample homogeneity found in other cell lysing and homogenization processes translates into poor quantitative results with respect to biomolecule recover and sample transfers that are not reproducible.

In one embodiment, focused acoustic energy may be used to lyse whole blood cells, as well as homogenize the cell lysate. For example, focused acoustic energy may be used to shear genomic DNA so that all of the genomic DNA is sheared to DNA fragments that are 50% or less of the starting base pair size of the genomic DNA. Since the viscosity of a cell lysate is frequently due at least in part to high molecular weight DNA and associated compounds, shearing of the genomic DNA can substantially reduce the viscosity of the sample, allowing for better or more homogenous mixing of the cell lysate and automated pipetting or other transfer operations. Moreover, blood cells can be lysed and genomic DNA sheared in a single vessel by focused acoustic energy, i.e., genomic DNA can be sheared in the presence of other cell lysate components. Prior shearing methods required genomic DNA to be separated from other cell lysate components to achieve suitable shearing. In embodiments of the invention, such pre-shearing separation of DNA can be avoided entirely.

The inventors have found that focused acoustic energy can speed or otherwise reduce the time needed to properly lyse cells and homogenize cell lysate, as well as provide a standardized or normalized result with respect to cell lysate viscosity and biomolecule recovery rates. Good homogenization of the resulting lysate by focused acoustic energy facilitates the direct purification of nucleic acid material, e.g., using a magnetic bead-based matrix. This is in contrast to other cell lysing and homogenization techniques which do not properly homogenize lysate, resulting in magnetic beads clumping together and compromising DNA yield. Also, lysing techniques that employ a detergent in the lysing buffer facilitates release of chromatin, which is highly viscous if not properly homogenized. Focused acoustic energy has been found to provide proper shear forces to suitably shear or otherwise disrupt chromatin to reduce sample viscosity and provide a uniformly homogenized lysate. Proper homogenization of whole blood lysate or other cell lysates enables direct transfer of small, nanoliter scale droplets, e.g., via acoustic levitation (LabCyte) or small volume pipetting. In the absence of the type of homogenization provided by focused acoustic energy, such transfer is not reproducible, since the analyte (e.g., DNA entangled in cell debris and proteins) is not evenly distributed in the lysate.

Focused acoustic energy has also been found to simplify the cell lysis and DNA isolation workflow when dealing with whole blood. For example, magnetic beads can be present during focused acoustic energy lysis without detrimental impact on biomolecule yield. The same is not true of other lysing techniques, such as vortexing. Thus, when using focused acoustic energy, after lysing the blood cells, a binding reagent (e.g., salt, crowding agent) may be added, followed by magnetic separation of the DNA bound to the beads. After washing the beads, pure DNA is eluted. All of this processing, cell lysis, homogenization, etc. can be done in a single vessel while allowing for high recovery of DNA.

### Example 1 (Blood Lysis)

Two parallel tests were performed to compare results of DNA isolation from 25 ul (microliters) of whole blood (healthy donor blood collected in K3-EDTA). In a first test, the whole blood was lysed using a standard vortexing method, i.e., the blood was mixed in a 1.7 ml microcentrifuge tube with 95 ul of lysis buffer (10 mM Tris, pH 8.0, 1 mM EDTA, 0.01% Brij-58) and 5 ul of magnetic beads (SpeedBeads magnetic carboxylate modified). The solution was then vortexed on a tabletop vortexer (VWR) for 90 seconds at room temperature. In a second test, the whole blood was lysed using focused acoustic energy. In the second test, the blood was provided into a microTUBE-130 (Covaris PN520216) and treated on an LE220 Focused-ultrasonicator (Covaris PN500569) for 90 seconds at 275 Watts peak incident power (PIP), 25% duty factor (% DF), 1000 cycles per burst (CpB) at 20 degrees C. (water bath setting for the acoustic coupling medium). The lysate resulting in both tests was then diluted with 325 ul of PK buffer (2.25% SDS, 50 mM Tris, pH 8.0, 20 mg/ml proteinase K) and incubated at 56 degrees C. for 30 min. All lysates were then mixed by vortexing with 245 ul Binding Solution (4M GITC, 2.4M NaCl, 100 mM Tris-HCl pH 7.5, 4% Triton X-100) and 560 ul of 100% isopropanol and incubated at room temperature for 5 min. The samples were then placed on a magnet for 5 min (until the supernatant was clear) and then the supernatant was removed without disturbing the beads. The solution recovered from each test then underwent a series of washes: 1) 350 uL of 1.6M GITC, 0.6M NaCl, 0.033M TrisHCL pH 7.5, 0.5% Triton X-100, 2) 350 uL of 0.6M NaCl, 0.033M TrisHCL pH 7.5, 0.5% Triton X-100, 3) 350 uL of freshly made 80% ethanol, 4) 350 uL of freshly made 80% ethanol. Between each wash, each sample was placed on a magnet until the supernatant was clear and then the supernatant was removed. The samples were air dried for 5 minutes and then eluted in 5 mM TrisHCL at pH 8.5. DNA recovery analysis was performed on the Qubit (Broad Range) and fragment analyzer (HS Large Fragment Gel) according to the manufacturer's instructions.

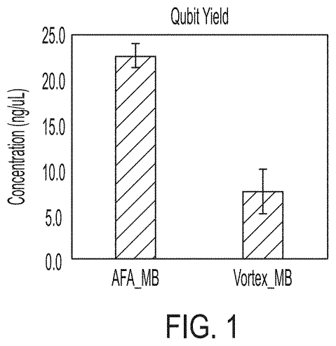

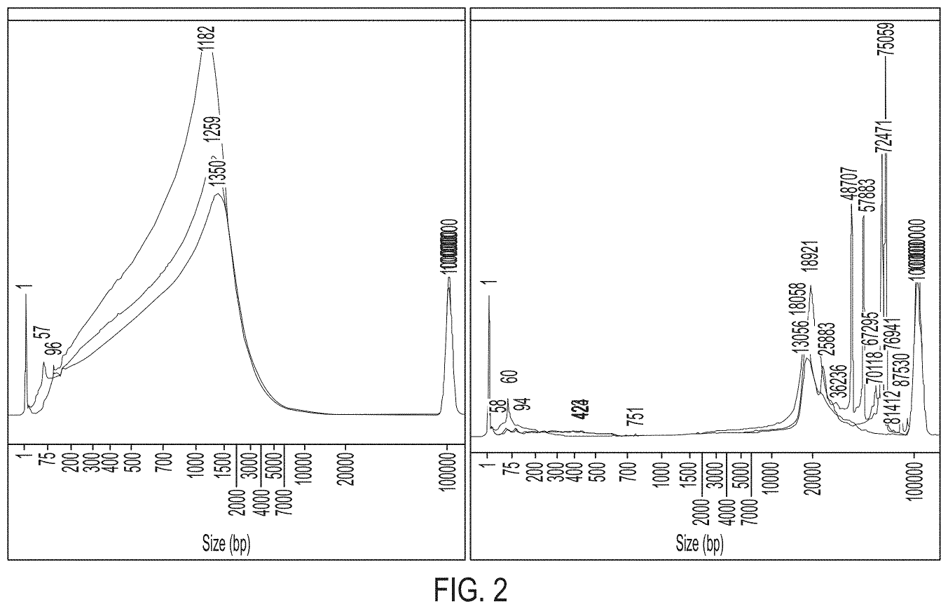

DNA yields obtained following lysis/homogenization with the focused acoustic energy method were found to be significantly higher compared to the yields obtained with the Vortex method. FIG. 1 shows the concentration of DNA recovered from each test, with the sample treated with focused acoustic energy indicated by “AFA_MB” and the vortexed sample indicated by “Vortex_MB.” As can be seen, the concentration of DNA in the focused acoustic energy treated sample is about 3 times higher than for the sample treated by vortexing. It is believed that these results are due in part by better bead binding kinetics in the focused acoustic energy lysed/homogenized sample as compared to the vortexed sample. Indeed, the vortexed sample was found to prevent proper magnetic bead separation, magnetic bead washing, and DNA elution, e.g., because of the relatively high viscosity of the sample after vortexing. DNA fragment length was also reduced to an average of 1.2 kbp in the focused acoustic energy treated sample as compared to the very heterogeneous higher molecular weight DNA in the Vortex treated sample. FIG. 2 shows the electropherograms of the DNA isolated for each of the two test samples and illustrates how the focused acoustic energy treated sample has significantly smaller fragment sizes than the vortex treated sample. (Fragment Analyzer (AATI) electropherograms). The focused acoustic energy treated sample results are on the left in FIG. 2; the vortexed sample results on the right.) The lower fragment sizes in the focused acoustic energy treated sample helps to reduce viscosity of the sample while increasing homogeneity of the lysate. The focused acoustic energy treated sample and resulting homogenization also impacts the purity of the isolated DNA, as expressed by the A260/280 ratios. These were found to be 1.8 (+/−0.03) and 1.6 (+/−0.04) for the focused acoustic energy and Vortex samples, respectively.

### Example 2 (Blood Lysis in the Covaris oneTUBE)

To demonstrate that focused acoustic energy treatment normalizes an extractable cell lysate (i.e., focused acoustic energy treats cell lysate so as to have a relatively constant and lower viscosity throughout to enable automated pipetting in a consistent and reliable way), seven samples of a highly concentrated whole blood lysate were subjected to extraction and purification of DNA. The extraction vessel in each case was a single tube in a 96 oneTUBE-10 AFA Plate (Covaris PN 520249). For each sample, 25 ul of whole blood (EDTA stabilized) was mixed in a oneTUBE-10 vessel with 25 ul of lysis buffer (10 mM Tris, pH 8.0, 1 mM EDTA, 0.01% Brij-58), 5 ul of magnetic beads (SpeedBeads magnetic carboxylate modified) and 5 ul of proteinase K solution (20 mg/ml). The solution for six of the samples was then subjected to different levels of focused acoustic energy on an LE220-Plus Focused-ultrasonicator (Covaris PN500569) at 425 W or 325 W Peak Incidence Power (PIP), 50% Duty Factor, 200 Cycles per Burst for 1, 11 and 22 discrete 2 second bursts interspersed by 1 second pauses of focused acoustic energy (either PIP=425 W or 325 W with 25% Duty Factor and 200 CpB). Temperature setting for the waterbath (acoustic coupling medium) was 20 degrees C. for all acoustic energy treated samples. The seventh sample was simply treated by pipetting up and down ten times during lysis (a recommended treatment step in at least some chemical lysis processes). However, this seventh sample was too viscous for magnetic bead clean-up even after adding the Bind Buffer 3 (see below), preventing proper Magnetic Bead separation in the lysate. This was found to impact the total yield of extracted DNA.

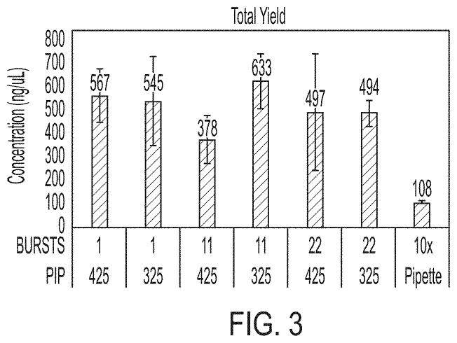

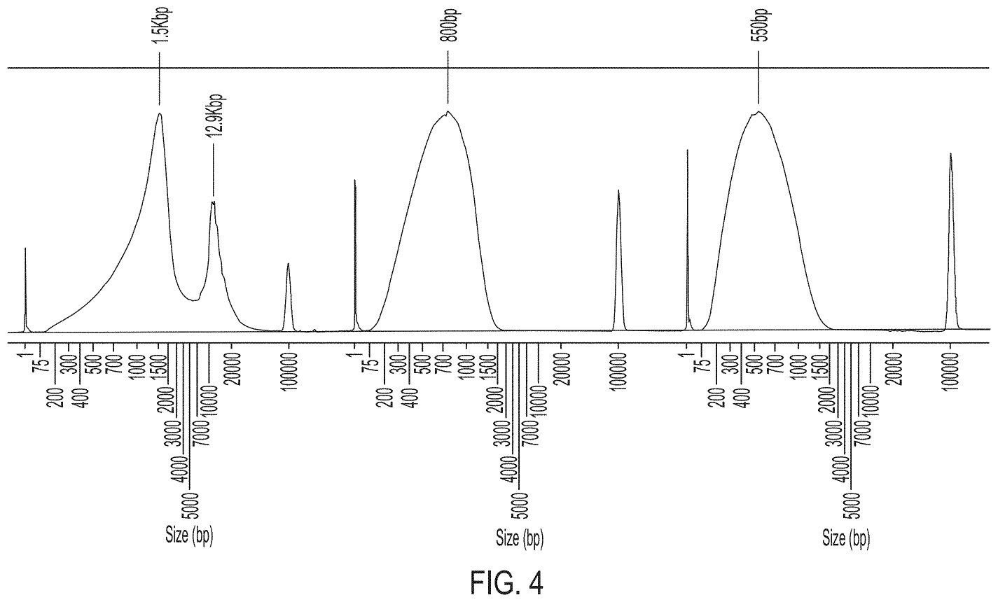

Following focused acoustic energy treatment, or mixing by pipetting, the lysate for each of the seven samples was then incubated for 15 min at 56 degrees C., followed by addition of 1.2× volumes of 11.6% PEG 8000, 0.6M NaCl, 20 mM Tris-HCl pH 8, 0.1% Triton-X, 0.001% Antifoam A (Bind Buffer 3). The samples were placed on a magnet for 5 min (until the supernatant was clear) and then the supernatant was carefully removed without disturbing the beads. The solution for each sample then underwent a series of washes: 1) 150 ul of 1.6M guanidinium thiocyanate, 0.6M NaCl, 0.033M Tris HCL pH 7.5, 0.5% Triton X-100, 2) 150 uL of 0.6M NaCl, 0.033M Tris HCL pH 7.5, 0.5% Triton X-100, 3) 150 uL of freshly made 80% ethanol, 4) 150 uL of freshly made 80% ethanol. Between each wash, the sample was placed on magnet until the supernatant was clear and then the supernatant was removed. The samples air dried for 5 minutes and were then eluted in 50 ul 5 mM TrisHCL at pH 8.5. Analysis was performed on the Qubit (Broad Range, ThermoFisher) and fragment analyzer (HS Large Fragment Gel, AATI) according to the manufacturer's instructions. FIG. 3 depicts the total yield of DNA and FIG. 4 shows the corresponding electropherograms of the fragment size distribution for three samples (from left to right in FIG. 4) that were treated with focused acoustic energy at a PIP of 325 W for 1, 11 and 22 bursts, respectively. Resulting DNA fragment size generally reduces with increased total treatment time with focused acoustic energy. As expected the DNA is sheared proportionally to the amount of the total energy dose applied. The single burst treatment sample (far left in FIG. 4) is showing two DNA fragment length populations, i.e., one with high molecular weight Mode (12.9 kb) and one with low molecular weight Mode (1.5 kb) pool. The samples homogenized with 11 and 22 AFA bursts (center and right plots in FIG. 4) show fragment distribution Modes of 800 bp and 550 bp, respectively. Note, that the sample that was homogenized by pipetting, has a fragment size Mode of >30 kb is not shown.

### Example 3 Lysis of Yeast Cells

DNA was isolated from three samples of 3×10⁷ cells of Y. lipolytica cultured overnight to the exponential growth phase. Cells were pelleted at >2000×g for 5 minutes before re-suspending to 30 uL with Covaris Lysis buffer. The samples were then aliquoted into the wells of a 96 oneTUBE-10 AFA plate (Covaris PN 520249) and treated on an LE220-plus Focused-ultrasonicator (Covaris PN 500569) for 5 to 15 minutes per row at 450 Watts PIP, 50% duty factor, 200 cycles per burst (10 degree C. water bath) to lyse the yeast cells. The lysate was treated with 2 uL of Proteinase K by incubating at 56 C for 15 minutes. Samples were then mixed with 13.7 uL 4 M guanidine thiocyanate, 100 mM Tris pH 7.5, 2.4 M NaCl, 4% Triton X-100 by vortexing, followed by 5 uL magnetic beads and 33 uL 100% isopropanol. The oneTUBE plate was placed on a magnet, and beads allowed to settle for 5 minutes. Supernatant was removed and discarded before removing the plate and resuspending beads in 200 uL 1.6 M guanidinium thiocyanate, 33 mM Tris pH 7.5, 0.6 M NaCl, 0.5% Triton X-100. The oneTUBE plate was again placed on a magnet, and beads allowed to settle for 5 minutes. Supernatant was discarded and 150 uL 80% ethanol was added to each well without disturbing the beads. Following a 30 second incubation, supernatant was discarded, and the ethanol wash repeated. All remaining ethanol was removed, and beads allowed to dry for 15 minutes before removing the plate from the magnet and re-suspending in either TE or water. The oneTUBE plate was returned to the magnet and incubated for 1 minute before transferring purified DNA to a clean vessel for storage.

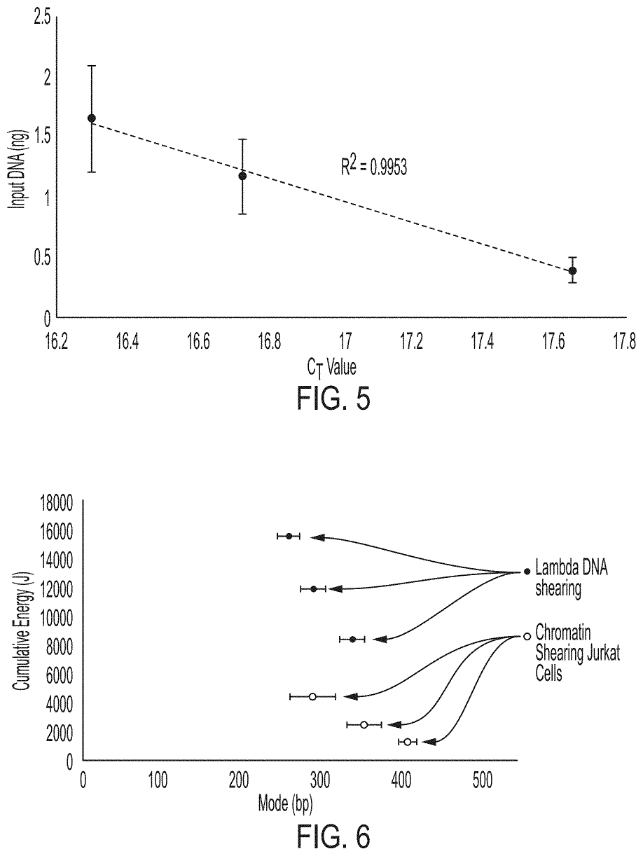

Following purification, samples were treated with 1 uL RNase A and incubated at 37 degrees C. for 30 minutes. DNA quantification was performed via Qubit (Thermo Fisher PN Q32854) and qPCR. PCR primers targeted the Eukaryotic 18 s sequence and were obtained from Thermo Fisher Scientific (Thermo Fisher PN 4352407) and thermocycling parameters were followed as recommended by the manufacturer. As shown in FIG. 5, Ct values obtained correlate well (R2=0.9953) with DNA input obtained at different focused acoustic treatment times.

### Example 4 DNA Shearing with and without Presence of Cell Lysate

Experiments were done to determine if focused acoustic energy DNA shearing in the presence of cell lysate provides different results than when done using isolated DNA. Surprisingly, the inventors have found that focused acoustic energy shearing of genomic DNA is different when done in the presence of cell lysate as compared to when done with isolated DNA. Specifically, shearing of isolated DNA in a solution follows different kinetics as compared to shearing DNA that is wrapped and packed into a cellular complex (such as chromatin). As an example, when chromatin from formalin-fixed cells is sheared by focused acoustic energy, the energy needed to fragment the DNA to a mode of 350 bp is significantly lower as compared to the energy needed to shear isolated DNA.

In this example, samples including Jurkat cells were lysed and the cell lysate homogenized and genomic DNA sheared using focused acoustic energy. Other samples included only isolated DNA, specifically Lambda DNA. All samples were treated with focused acoustic energy in Covaris Shearing Buffer using a LE220 instrument. The Lambda DNA samples used were ˜5 ng/μl, while the number of cells were about 1M cells/treatment well. The volume used for shearing was 50 μl for all test samples (N=16 for Lambda DNA and N=8 for Jurkat Cells). The settings used on the LE220 instrument for this test were 200 PIP, 20% DF, 50 CPB, with different treatment times. The treatment times tested for Lambda DNA were 210 seconds, 300 seconds, and 390 seconds. The treatment times tested for Jurkat cells were 30 seconds, 60 seconds, and 110 seconds. Significantly lower energy was needed (about 10%-20%) to achieve a similar mode of around 300 bp for sheared genomic DNA fragments with Jurkat cells than was needed to shear Lambda DNA in the same buffer. FIG. 6 shows the cumulative energy versus fragment mode for the samples. Generally, Lambda DNA required two to four or more times the cumulative energy to achieve a similar fragment mode.

This example shows that focused acoustic energy can be very useful for lysing and shearing genomic DNA, and particularly chromatin. The sheared chromatin can be used in various downstream analyses, such as Chromatin-Immunoprecipitation Assay (ChIP) which involves the lysis of formalin-fixed or native eukaryotic cells, followed by the fragmentation of the chromatin and specific binding of target proteins such as histones, transcription factors and other DNA associated proteins to magnetic bead-conjugated antibodies (Immuno Precipitation). The associated DNA is then released by de-crosslinking or salt-based dissociation (native ChIP) and analyzed by sequence analysis, qPCR etc. Chromatin released from eukaryotic nuclei generally form a viscous matrix that is difficult to pipette, therefore seriously compromising and even preventing aliquotation and liquid transfer steps. As noted above, this often prevents automated handling of chromatin material. However, the inventors have found that focused acoustic energy homogenizes the released chromatin by fragmenting the genomic DNA and thereby reducing the viscosity of the sample. Controlled fragmentation achieved by the focused acoustic energy ensures that protein-DNA complexes are intact, which is a pre-requisite for proper antibody—antigen recognition. Since the antibodies in this assay are conjugated to magnetic beads, the viscosity of the lysate will adversely impact the kinetics of binding, thereby impacting sensitivity as well as specificity. Focused acoustic treatment of the cell lysate including chromatin avoids such problems.

### Example 5 Extraction and Parallel Purification of RNA and DNA from Cell Culture

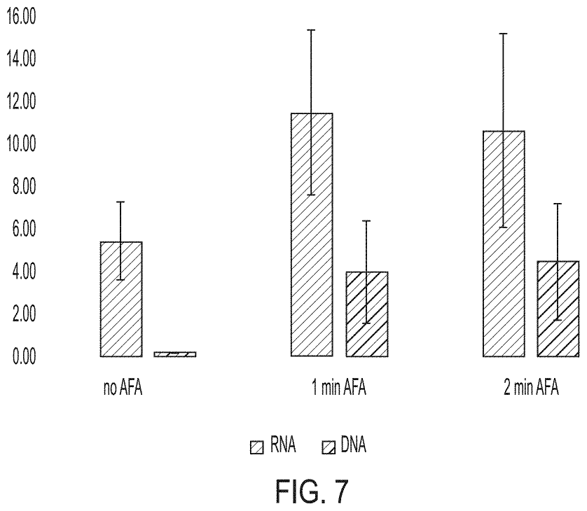

The impact of focused acoustic energy homogenizing of human cell culture lysate on purification of DNA and RNA was assessed with an off-the-shelf kit, i.e., the Invitrap Spin Cell RNA Mini Kit (Stratec Molecular). The commercial use of these kits currently is focused on only purifying DNA-free RNA, i.e., the DNA containing matrix is discarded. In other words, these kits have not been found suitable for purifying DNA. However, the inventors have found that when the kit is used in conjunction with focused acoustic energy, the DNA yield is dramatically increased. For this experiment, 500 ul of a human cell culture suspension containing approximately 2.5 million cells was collected by centrifugation and the pelleted cells were suspended in 400 ul Lysis Buffer R (provided in the kit). The lysate was treated in two ways: either with focused acoustic energy (PIP=350 W, DF=25%, CpB=1000) for 1 or 2 minutes or processed as instructed in the kit manual. The lysate from each sample was then filtered through the supplied filter cartridge. According to the information from the kit supplier, the DNA is captured to a fine glass bead matrix during the lysis process, whereas the RNA does not bind to this matrix under the same buffer (lysis Buffer R) conditions. The DNA-containing matrix is then removed by filtering. The flow through material contains DNA-free RNA which is in then further treated by adding a binding buffer and retrieving/eluting the RNA on/from a separate filter. In this experiment, the DNA captured in that filter was, contrary to the kit manual instructions, not discarded, but, like the RNA, purified by washing the filter using the supplied wash buffers and then eluted in TE buffer. The RNA in the flow though lysate was isolated, washed and eluted as described in the kit manual. Focused acoustic energy treatment during the lysis step dramatically increased the yield of DNA as compared to the non-focused acoustic energy treated sample by more than 20 fold, as can be seen in FIG. 7. The yield of the RNA is also increased by 2 fold as shown in FIG. 7. This effect is believed to be due to the homogenization of the lysate, thereby releasing and normalizing the analytes of interest (DNA and RNA) and making them accessible for the purification process.

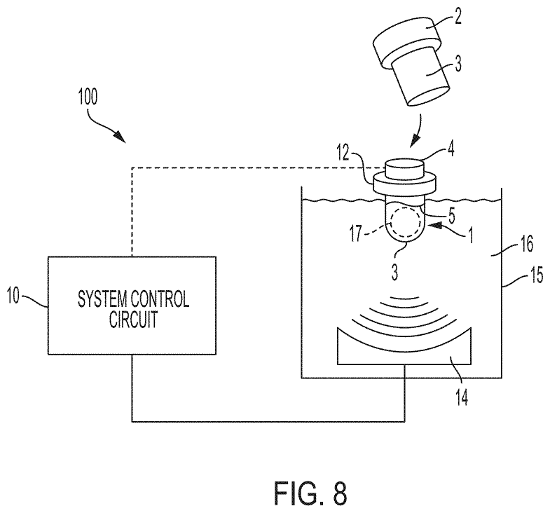

FIG. 8 shows a schematic block diagram of an acoustic treatment system 100 that incorporates one or more aspects of the present disclosure and/or can be employed with one or more aspects of the described herein. It should be understood that although embodiments described herein may include most or all aspects of the invention(s), aspects of the invention(s) may be used alone or in any suitable combination with other aspects of the invention(s).

In this illustrative embodiment, the acoustic treatment system 100 includes an acoustic energy source with an acoustic transducer 14 (e.g., including one or more piezoelectric elements) that is capable of generating an acoustic field (e.g., at a focal zone 17) suitable to cause mixing, e.g., caused by cavitation, and/or other affects on a sample contained in a vessel 4. The sample may include solid particles or other material on a porous element, and/or liquid material in the vessel. Acoustic energy may be transmitted from the transducer 14 to the vessel 4 through a coupling medium 16, such as a liquid (e.g., water), a gel or other semi-solid, or a solid, such as a silica, metal or other material. Thus, the transducer 14 is spaced from the vessel 4 and can transmit acoustic energy from outside the vessel volume for transmission into the vessel 4 via the coupling medium 16. Where the coupling medium 16 is a liquid, a coupling medium container 15 may be used to hold the coupling medium 16.

The vessel 4 may have any suitable size or other arrangement, e.g., may be a glass or metal tube, a plastic container, a well in a microtiter plate, a vial, or other, and may be supported at a location by a vessel holder 12. Although a vessel holder 12 is not necessarily required, the vessel holder 12 may interface with the control circuit 10 so that the vessel 4 and the sample in the vessel is positioned in a known location relative to an acoustic field, for example, at least partially within a focal zone 17 of acoustic energy. In this embodiment, the vessel 4 is a 130 microliter borosilicate glass tube, but it should be understood that the vessel 4 may have other suitable shapes, sizes, materials, or other feature, as discussed more below. For example, the vessel 4 may be a cylindrical tube with a flat bottom and a threaded top end to receive a cap 2, may include a cylindrical collar with a depending flexible bag-like portion to hold a sample, may be a single well in a multiwell plate, may be a cube-shaped vessel, or may be of any other suitable arrangement. The vessel 4 may be formed of glass, plastic, metal, composites, and/or any suitable combinations of materials, and formed by any suitable process, such as molding, machining, stamping, and/or a combination of processes.

The transducer 14 can be formed of a piezoelectric material, such as a piezoelectric ceramic. In some embodiments, the ceramic may be fabricated as a “dome,” which tends to focus the energy. One application of such materials is in sound reproduction; however, as used herein, the frequency is generally much higher and the piezoelectric material would be typically overdriven, that is driven by a voltage beyond the linear region of mechanical response to voltage change, to sharpen the pulses. Typically, these domes have a longer focal length than that found in lithotriptic systems, for example, about 20 cm versus about 10 cm focal length. Ceramic domes can be damped to prevent ringing or undamped to increase power output. The response may be linear if not overdriven. The high-energy focus zone of one of these domes may be cigar-shaped. At 1 MHz, the focal zone is about 6 cm long and about 2 cm wide for a 20 cm dome, or about 15 mm long and about 3 mm wide for a 10 cm dome. The peak positive pressure obtained from such systems is about 1 MPa (mega Pascal) to about 10 MPa pressure, or about 150 PSI (pounds per square inch) to about 1500 PSI, depending on the driving voltage. The focal zone 17, defined as having an acoustic intensity within about 6 dB of the peak acoustic intensity, is formed around the geometric focal point. It is also possible to generate a line-shaped focal zone, e.g., that spans the width of a multi-well plate and enables the system 100 to treat multiple samples simultaneously. Other arrangements for producing focused acoustic energy are possible. For example, a flat transducer may be provided with a tapered waveguide for focusing or otherwise channeling acoustic energy emitted from the transducer toward a relatively small space where the sample and vessel are located.

To control an acoustic transducer 14, the acoustic treatment system 100 may include a system control circuit 10 that controls various functions of the system 100 including operation of the acoustic transducer 14. For example, the system control circuit 10 may provide control signals to a load current control circuit, which controls a load current in a winding of a transformer. Based on the load current, the transformer may output a drive signal to a matching network, which is coupled to the acoustic transducer 14 and provides suitable signals for the transducer 14 to produce desired acoustic energy. Moreover, the system control circuit 10 may control various other acoustic treatment system 100 functions, such as positioning of the vessel 4 and/or acoustic transducer 14, receiving operator input (such as commands for system operation), outputting information (e.g., to a visible display screen, indicator lights, sample treatment status information in electronic data form, and so on), and others. Thus, the system control circuit 10 may include any suitable components to perform desired control, communication and/or other functions. For example, the system control circuit 10 may include one or more general purpose computers, a network of computers, one or more microprocessors, etc. for performing data processing functions, one or more memories for storing data and/or operating instructions (e.g., including volatile and/or non-volatile memories such as optical disks and disk drives, semiconductor memory, magnetic tape or disk memories, and so on), communication buses or other communication devices for wired or wireless communication (e.g., including various wires, switches, connectors, Ethernet communication devices, WLAN communication devices, and so on), software or other computer-executable instructions (e.g., including instructions for carrying out functions related to controlling the load current control circuit as described above and other components), a power supply or other power source (such as a plug for mating with an electrical outlet, batteries, transformers, etc.), relays and/or other switching devices, mechanical linkages, one or more sensors or data input devices (such as a sensor to detect a temperature and/or presence of the medium 16, a video camera or other imaging device to capture and analyze image information regarding the vessel 4 or other components, position sensors to indicate positions of the acoustic transducer 14 and/or the vessel 4, and so on), user data input devices (such as buttons, dials, knobs, a keyboard, a touch screen or other), information display devices (such as an LCD display, indicator lights, a printer, etc.), and/or other components for providing desired input/output and control functions.

Under the control of a control circuit 10, the acoustic transducer 14 may produce acoustic energy within a frequency range of between about 100 kilohertz and about 100 megahertz such that the focal zone 17 has a width of about 2 centimeters or less. The focal zone 17 of the acoustic energy may be any suitable shape, such as spherical, ellipsoidal, rod-shaped, or column-shaped, for example, and be positioned at the sample. The focal zone 17 may be larger than the sample volume, or may be smaller than the sample volume, as shown in FIG. 8. U.S. Pat. Nos. 6,948,843 and 6,719,449 are incorporated by reference herein for details regarding the construction and operation of an acoustic transducer and its control.

Samples processed according to embodiments may include a solution or mixture that is suitable for lysing blood cells and extracting target biomolecules (e.g., DNA, RNA, protein, etc.). In some embodiments, the liquid in the vessel 4 includes a buffer, such as water along with a detergent, e.g., a 0.25% SDS (sodium dodecyl sulfate), Triton-X100, Tween-20, Brij-58, NDSB (non-detergent sulfobetaine), zwitterionic surfactant, such as CHAPS, CHAPSO, Zwittergent, etc. solution, although other solutions are possible, such as ethanol organic solvent/water mixtures (e.g., 10 to 50% acetonitrile) and/or other suitable buffered solutions, an enzyme-containing solution, etc.

After a suitable degree of focused acoustic treatment, as noted above, various enzymes or other agents may be activated and/or kinetically enhanced accelerated, resulting in the removal of certain molecules or contaminants from the mixture. For instance, as noted above, when seeking to recover nucleic acids from the sample, it may be preferable to include Proteinase K within the mixture, and accelerating enzymatic activity thereof by adjusting the temperature of the sample to approximately 56 degrees C. and by intermittently or continuously treating the solution with focused acoustic energy.

In an embodiment where the acoustic treatment system 100 is a Covaris device, acoustic treatment may be applied using a duty cycle, a peak incident power (PIP), cycles per burst (CPB), for a suitable period of time as discussed above. Of course, other duty cycles, peak power, cycles per burst and/or time periods may be used to produce a sufficient amount of power for processing different samples. For example, to achieve desirable results with regard to extraction and recovery of biomolecules from a sample and with regard to quality of the extracted biomolecules, the acoustic transducer may be operated at a peak intensity power of between 100 W and 300 W, a duty factor of between 10% and 90% and a cycles per burst of between 100 and 1000, for an appropriate duration of time. It can be appreciated that the acoustic transducer may be operated so as to produce focused acoustic energy that results in a suitable level of energy input to the sample material.

In some embodiments, the transducer may generate acoustic energy having a peak incident power over the course of a period of time that produces a particular amount of energy, to achieve preferred results. As described herein, the peak incident power (PIP) is the power emitted from the transducer during the active period of one cycle. The peak incident power, in some cases, may control the amplitude of the acoustic oscillations. The energy applied to the sample material may be determined from the peak incident power of the applied acoustic energy and the duration of the acoustic treatment period. In some embodiments, to suitably lyse cells and extract or otherwise operate on the target biomolecule(s) from a sample, the acoustic transducer may be operated so as to generate focused acoustic energy according to a peak incident power of greater than or equal to 50 Watts, greater than or equal to 100 Watts, greater than or equal to 150 Watts, greater than or equal to 200 Watts, greater than or equal to 250 Watts, greater than or equal to 300 Watts, or other values outside of these ranges.

The acoustic transducer may be operated at a suitable duty factor, in combination with other parameters, to generate focused acoustic energy that leads to preferred results. As described herein, the duty factor is the percentage of time in a cycle in which the transducer is actively emitting acoustic energy. For example, a duty factor of 60% refers to the transducer being operated in an “on” state 60% of the time, and in an “off” state 40% of the time. In some embodiments, in appropriately lysing cells and extracting/processing the target biomolecule(s) from a sample, the acoustic transducer may be operated at a duty factor setting of greater than or equal to 10%, greater than or equal to 20%, greater than or equal to 30%, greater than or equal to 40%, greater than or equal to 50%, greater than or equal to 60%, greater than or equal to 70%, or greater than or equal to 80%, or other values outside of these ranges.

The acoustic transducer may be operated according to a suitable cycles-per-burst setting to achieve preferred results. As described herein, the cycles per burst (CPB) is the number of acoustic oscillations contained in the active period of one cycle. In some embodiments, to lyse and extract/process the target biomolecule(s) from a sample, the acoustic transducer may be operated to generate focused acoustic energy according to a cycles per burst setting of greater than or equal to 50, greater than or equal to 100, greater than or equal to 150, greater than or equal to 200, or other values outside of these ranges.

After a suitable degree of focused acoustic treatment, as noted above, various enzymes or other agents may be activated and/or accelerated, resulting in the removal of certain molecules or contaminants from the mixture. For instance, as noted above, when seeking to recover nucleic acids from the sample, it may be preferable to include Proteinase K within the mixture, and accelerating enzymatic activity thereof by adjusting the temperature of the sample to approximately 56 degrees C. However, such proteinase need not be employed, particularly before cell lysing and shearing of genomic DNA is complete.

It should also be appreciated that for various embodiments, the duration of time in which the sample is subject to focused acoustic treatment may affect the average DNA fragment size. That is, the focused acoustic processing not only provides for extraction of the target biomolecules (i.e., nucleic acids), but also for shearing and fragmenting thereof. For example, the average DNA fragment size may decrease as the duration of acoustic energy treatment is increased and, conversely, the resulting average DNA fragment size may be larger for shorter durations of acoustic energy treatment. The average DNA fragment size may be tuned according to the duration of time under which the sample is exposed to a suitable amount of focused acoustic energy.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

While aspects of the present disclosure have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the present disclosure.

What is claimed is:

1. A method for recovering biomolecular constituents from whole cells, comprising:
providing a sample in a vessel including a plurality of whole cells;
exposing the sample to a focal zone of focused acoustic energy to lyse the plurality of whole cells to release cell lysate from the plurality of whole cells, the cell lysate including genomic DNA having a starting base pair size; and
exposing the cell lysate to a focal zone of focused acoustic energy to shear the genomic DNA in the cell lysate such that all the genomic DNA is sheared to DNA fragments that have a fragment size no larger than 50% of the starting base pair size and so that a viscosity of the cell lysate is reduced.

2. The method of claim 1, wherein the steps of providing the sample, exposing the sample and exposing the cell lysate are performed in a single vessel without removing the plurality of whole cells, cell lysate or genomic DNA from the single vessel.

3. The method of claim 1, wherein the sample includes whole blood or a tissue sample.

4. The method of claim 1, wherein providing a sample includes providing a detergent solution in the vessel.

5. The method of claim 1, wherein the steps of providing the sample, exposing the sample, and exposing the cell lysate are performed without centrifuging any portion of the sample or cell lysate.

6. The method of claim 1, wherein the steps of providing the sample, exposing the sample, and exposing the cell lysate are performed without the addition of any enzyme to the sample or cell lysate.

7. The method of claim 1, further comprising:
separating the DNA fragments from other portions of the cell lysate.

8. The method of claim 7, wherein the steps of providing the sample, exposing the sample, exposing the cell lysate and separating the DNA fragments are performed without centrifuging any portion of the sample or cell lysate.

9. The method of claim 7, wherein the steps of providing the sample, exposing the sample, exposing the cell lysate and separating the DNA are performed with binding beads present with the sample and the cell lysate.

10. The method of claim 7, wherein the step of separating the DNA fragments includes diluting the cell lysate and DNA fragments with a buffer and a proteinase, incubating the cell lysate, DNA fragments, buffer and proteinase, and exposing the cell lysate, DNA fragments, buffer and proteinase to a magnetic field to separate DNA fragments bound to magnetic beads from other portions of the sample.

11. The method of claim 1, further comprising:
separating RNA fragments in the cell lysate from other portions of the cell lysate.

12. The method of claim 11, wherein the steps of providing the sample, exposing the sample, exposing the cell lysate and separating the RNA fragments are performed without centrifuging any portion of the sample or cell lysate.

13. The method of claim 11, wherein the steps of providing the sample, exposing the sample, exposing the cell lysate and separating the DNA are performed with binding beads present with the sample and the cell lysate.

14. The method of claim 1, wherein exposing the cell lysate includes shearing the genomic DNA such that the DNA fragments have a fragment size that is no larger than 50 kbp.

15. The method of claim 1, wherein exposing the cell lysate includes shearing the genomic DNA such that the DNA fragments have a fragment size that is no larger than 10 kbp.

16. The method of claim 1, wherein exposing the cell lysate includes shearing the genomic DNA such that the DNA fragments have a fragment size that is no larger than 2 kbp.

17. The method of claim 1, wherein the starting base pair size is greater than 1 Mbp.

18. The method of claim 17, wherein the DNA fragment size is less than 500 kbp.

19. The method of claim 1, wherein the step of exposing the sample is preceded by a step of exposing the sample to chemical, physical-chemical, mechanical or a combination of such processes to lyse cells in the sample.

20. A method for recovering genomic material from whole cells, comprising:
providing a sample in a vessel including a plurality of whole cells;
exposing the sample to a focal zone of focused acoustic energy to lyse the plurality of whole cells to release cell lysate from the plurality of whole cells, the cell lysate including HMW DNA having a starting base pair size; and
exposing the cell lysate to a focal zone of focused acoustic energy to shear the HMW DNA in the cell lysate such that all the HMW DNA is sheared to DNA fragments that have a fragment size no larger than 50% of the starting base pair size and so that a viscosity of the cell lysate is reduced.
